(19) United States
(12) Patent Application Publication
(10) Pub. No.: US 2008/0224066 A1
(43) Pub. Date: Sep. 18, 2008

(54) Modular, High Volume, High Pressure Liquid Disinfection Using Uv Radiation

(75) Inventors: Gary Nolen, Bella Vista, AR (US); Joe Rheingans, Rogers, AR (US)

Correspondence Address: Mark Rogers;Safe Foods Corporation, 4801 North Shore Drive, North Little Rock, AR, 72118, US

(73) Assignee: SAFE FOODS CORPORATION, Little Rock, AR (US)

(21) Appl. No.: 10/542,793

(22) PCT Filed: Sep. 3, 2003
(86) PCT No.: PCT/US2003/028218

§ 371 Date: May 30, 2008

Related U.S. Application Data
(60) Provisional application No. 60/441,930, filed on Jan. 21, 2003.

(51) Int. Cl. G01N 23/12

(52) U.S. Cl. 250/436

(57) ABSTRACT

A device for exposure of a fluid to radiation comprising a tube (20) through which a fluid is caused to flow , a plurality of radiation sources (14), and a plurality of reflectors (48) to cause the radiation to be focused on the fluid.

This application claims the benefit of U.S. Provisional Application Ser. No. 60/441,930, filed on Jan. 21, 2003, the disclosure of which is incorporated herein by reference.

### BACKGROUND OF THE INVENTION

The present invention relates to liquid disinfection and, more particularly, to liquid disinfection using ultraviolet (UV) radiation.

It is known to use UV radiation to disinfect clear or opaque liquids such as water, including wastewater, juices, brines, marinades, beverages, and the like. A couple of examples include U.S. Patent Nos. 3,527,940 and 4,968,891, the disclosures of which are incorporated herein by reference. Using UV radiation to disinfect liquids offers many advantages that often make it a very attractive option as compared to other methods of disinfecting liquids. It will often provide for improved disinfection in a fast, simple, relatively inexpensive manner.

Still, prior equipment and methods of disinfecting liquids using UV radiation suffer from a number of disadvantages. For example, the relatively fragile nature of the equipment has placed undesirable limitations on the flow rates that may be treated and operating pressures that may be used. The relatively fragile nature of the equipment similarly limited pressures and flow rates that could be used for cleaning purposes, making it more difficult or impossible to provide the convenience of clean in place equipment. The effectiveness of UV radiation to disinfect a liquid diminishes rapidly, likely exponentially, with distance, so relying primarily upon turbulence in a liquid to provide for even, thorough disinfection of the liquid can be unreliable. Also, exposure times for desired levels of disinfection can often lead to the use of undesirably large equipment or the use of an undesirably large number of units of such equipment, adding to the cost of the system and taking up valuable floor space. In a typical prior art unit, a significant portion of the radiation emitted by the bulbs is not directed toward the liquid to be treated and is wasted, making inefficient use of the radiation and of the power consumed to generate the radiation. Prior cabinets or units used to provide UV disinfection of liquids also provided little or no flexibility in handling differing flow patterns, flow rates, and treatment times. Further, prior cabinets and units were difficult and time-consuming to service or repair, and typically required an entire cabinet or unit to be shut down and placed out of service for extended periods.

### SUMMARY OF THE INVENTION

It is therefore an object of the present invention to provide a system and method for treating a liquid with radiation that offers increased efficiency.

It is a further object of the present invention to provide a system of the above type that allows the flexibility of switching between parallel and series flow with minimal adjustments.

It is a still further object of the present invention to provide a system of the above type that provides a rugged system that may handle high pressures and flow rates.

It is a still further object of the present invention to provide a system of the above type which uses modular illumination units to allow for fast and easy replacement of bulbs or other components.

It is a still further object of the present invention to provide a system of the above type that makes highly efficient use of radiation generated by treating bulbs.

It is a still further object of the present invention to provide a system of the above type which provides for an extended treatment path without a corresponding increase in the length of the treatment chamber.

It is a still further object of the present invention to provide a system of the above type which provides for more even and thorough exposure of the liquid to be treated.

It is a still further object of the present invention to provide a system of the above type which provides for the convenience of fluid input and output at the same end of a treatment chamber.

Toward the fulfillment of these and other objects and advantages, a radiation treatment method and device are disclosed. The device comprises a treatment chamber and a radiation source, such as one or more UV bulbs, disposed in close proximity thereto. The treatment chamber has a header to which are connected coaxially aligned inner and outer tubes. The coaxially aligned tubes form an annulus area, and a static mixer defines a spiral liquid travel path through the annulus. An exit path is provided through the center of the inner tube and through the header. Input and output manifolds are provided, and adjacent treatment chambers may alternately be aligned and connected to provide for parallel or serial flow. Modular illumination units may be used in which two mirror image halves each have a bracket that supports and aligns reflectors and UV bulbs adjacent each treatment chamber.

### BRIEF DESCRIPTION OF THE DRAWINGS

The above brief description, as well as further objects, features and advantages of the present invention will be more fully appreciated by reference to the following detailed description of the presently preferred but nonetheless illustrative embodiments in accordance with the present invention when taken in conjunction with the accompanying drawings, wherein:

FIG. 1 is a sectional, side elevation view of a treatment chamber forming part of a radiation treatment device of the present invention;

FIG. 2 is a sectional, overhead view of a radiation treatment device of the present invention;

FIG. 3 is a partial, side elevation view of an alternate embodiment of a radiation treatment device of the present invention;

FIG. 4 is a is a partial, sectional, overhead view of an alternate embodiment of a radiation treatment device of the present invention;

FIG. 5 is an overhead, perspective view of a parallel flow alignment of a radiation treatment device of the present invention;

FIG. 6 is an overhead, perspective view of a series flow alignment of a radiation treatment device of the present invention;

FIG. 7 is a front elevation view of a cabinet for housing a radiation treatment device of the present invention; and

FIG. 8 is a partial, side elevation view of a cabinet for housing a radiation treatment device of the present invention.

### DETAILED DESCRIPTION OF THE PREFERRED EMBODIMENT

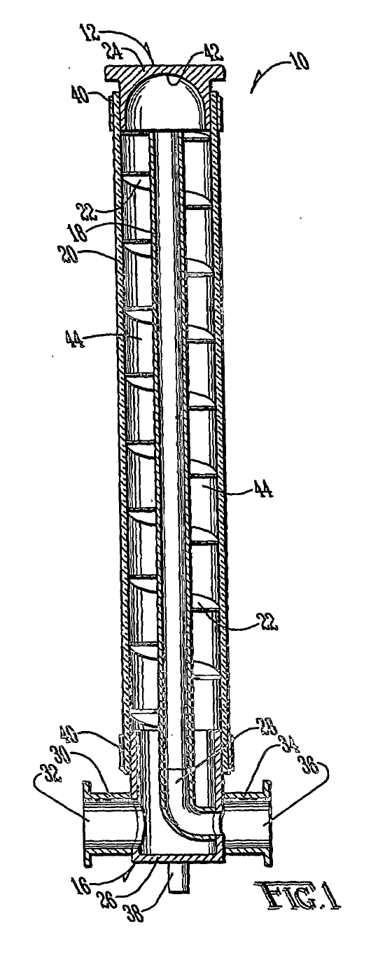

Referring to FIG. 1, the reference numeral 10 refers in general to a radiation treatment device of the above invention. The device 10 comprises a treatment chamber 12 and a radiation source 14 disposed in close proximity thereto.

The treatment chamber 12 comprises a header 16, inner and outer tubes 18 and 20, a static mixer 22, and an end cap 24. The header 16 has an outer housing 26, an inner header tube 28, an input pipe 30 with an input opening 32, and an output pipe 34 with an output opening 36. The outer housing 26 is open at the top, closed at the bottom, and has two side openings disposed on opposite sides, with one side opening being larger than the other. A mount 38 is secured to the bottom wall of the outer housing 26. The input pipe is affixed to the outer housing 26, aligned with the larger of the two side openings. The output pipe 34 is affixed to the outer housing 26 aligned with the smaller of the two other side openings. The input and output pipes 30 and 34 both have inner diameters of approximately 1.5 inches. The inner diameter of the output pipe 34 is larger than the diameter of the side opening. The inner header tube 28 has an input opening centrally disposed and coaxially aligned with the outer housing 26 and an output opening aligned with the smaller of the two side openings. The inner diameter of the inner header tube 28 is substantially the same as the diameter of this side opening. The header 16 is preferably made of stainless steel and is of clean in place construction. It is of course understood that the header 16 may be made of any number of different materials or combinations of materials. It is also understood that the header 16 may be assembled or fabricated from a number of different parts or may be cast or molded as one or more integral pieces.

Outer tube 20 is made of a material that is transparent to UV radiation or to the type of radiation used. The outer tube 20 is preferably constructed of a polymer, is more preferably constructed of a fluoropolymer, and is most preferably constructed of fluorinated ethylene propylene. The outer tube may of course be constructed of any number of materials known to possess the desired degree of transparency. The outer tube 20 has a length of approximately 60 inches and has an inner diameter of approximately 1.25 inches. A lower portion of the outer tube 20 is secured to the header 16, such as by using a hose clamp 40. The end cap 24 is affixed to an upper portion of the outer tube 20, such as by using a hose clamp 40. A lower surface 42 of the end cap 24 is curved to assist in redirection of the liquid with minimal pressure drop. The cap 24 is preferably stainless steel.

An output end of the inner tube 18 is affixed to the input end of the inner header tube 28, and the inner tube 18 extends coaxially aligned within the outer tube 20 along most if not all of the height of the outer tube 20. The inner tube 18 is preferably stainless steel having an inner diameter of substantially within a range of from approximately 0.5 inch to approximately 3.25 inch. The inner tube has an outer diameter that is substantially within a range of approximately from approximately 0.75 inch to approximately 3.5 inch. The outer diameter of the inner tube 18 and the inner diameter of the outer tube 20 are preferably selected to provide a relatively narrow annulus 44 between the two having a width of approximately 0.25 inch. An inner surface of the inner tube 18 defines an inner flow path. An inner surface of outer tube 20 and an outer surface of inner tube 18 define an outer flow path. An opening in a distal end of the inner tube 18 places the outer flow path in fluid flow communication with the inner flow path. The outer surface of the inner tube 18 is not transparent with respect to the radiation from the radiation source 14 and is preferably reflective of the radiation.

The static mixer or helical member 22 is an auger style static mixer that is affixed to the outer diameter of the inner tube 18, such as by welding. The mixer 22 extends between the outer wall of the inner tube 18 and the inner wall of the outer tube 20 and preferably contacts the inner wall of the outer tube 20. The mixer 22 is preferably stainless steel. Different degrees of winding may be used depending upon desired characteristics of the device 10. In one preferred embodiment the winding provides a liquid travel path of approximately 3.9 inches for each 1 inch of annulus 44 height. For a treatment chamber 12 in which the height of the annulus 44 area is approximately 60 inches, this would provide a liquid travel path of approximately 234 inches.

Referring to FIG. 2, a modular illumination unit 46 is provided, formed from two mirror image sections 47. The sections 47 are connected to one another by a hinge 49 or in any conventional manner. Each section 47 comprises a plurality of bulbs 14, one or more reflectors 48, and a bracket 50. The bracket 50 supports and aligns the bulbs 14 and supports and aligns the reflector or reflectors 48 positioned adjacent to the bulbs 14. The reflector 48 is configured with a curved portion or segment, such as a semi-circular, hyperbolic, or parabolic shaped portion or segment, associated with each bulb 14, disposed and aligned to reflect and focus radiation emitted from outer portions of the bulb 14 back toward the treatment chamber 12. The segments are disposed so that the reflector 48 is generally clamshell shaped. In that regard, a cross section of one segment falling in a common plane of a cross section of an adjoining segment does not form a portion of a common circle or semi-circle with the cross section of the adjoining segment. Each cross section is preferably semi-circular, and each cross section of a segment has an arc length that is greater than approximately 45°. The inner surface of the reflector 48 is selected to be highly reflective of the radiation used. For example, if a UV bulb 14 is used, the inner surface is preferably polished aluminum. Each section 47 is secured to its mating section 47 and is secured within the cabinet 66 in any number of ways, such as being secured to a back wall of the cabinet or to brackets disposed within the cabinet 66. In the preferred embodiment, one section 47 is disposed toward a back portion of the cabinet 66, and a mating section 47 is disposed toward a front portion of the cabinet 66 so that the front section 47 may be easily opened to provide access to the treatment chamber 12 and to the sections 47 of the illumination unit 46. Each section 47 is independently removable without the need to remove an associated treatment chamber or mated section 47. The brackets 50 of each section 47 are disposed to place the bulbs 14 in very close proximity to the outer surface of the outer tube 20. In the preferred embodiment, in which the modular concept is used, a separate modular illumination unit 46 is associated with each treatment chamber 12. It is also preferred to provide an extra or spare modular illumination unit 46 along with the device 10. This will reduce down time by making it easy to quickly replace an installed unit 46 with a spare unit 46 if the installed unit is in need of repair, maintenance, or replacement.

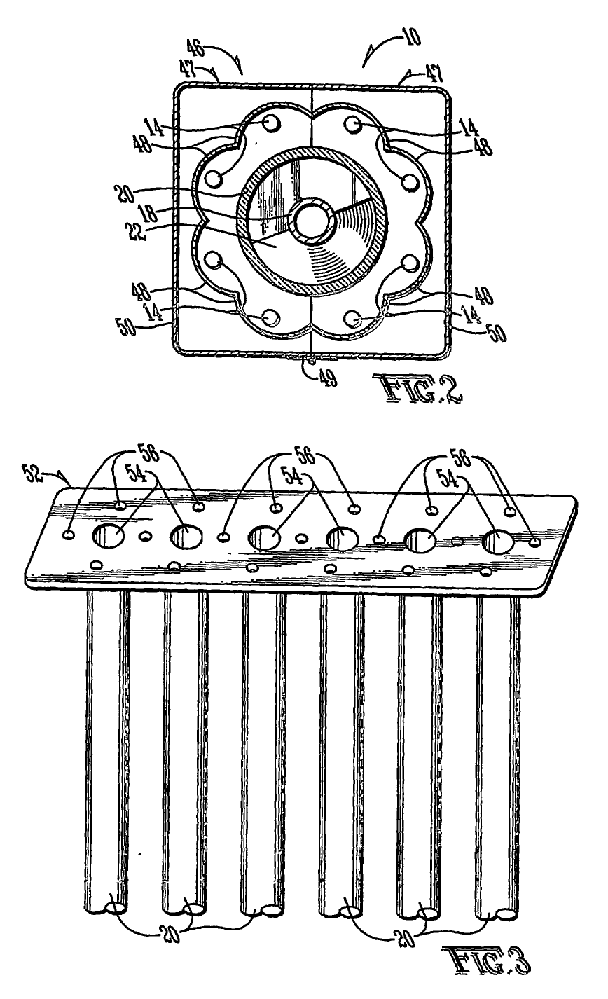

In an alternate embodiment depicted in FIGS. 3 and 4, one or more bulb racks 52 may be used to support and align a plurality of outer tubes 20 of a plurality of treatment chambers 12, along with the bulbs 14 and reflectors 48 to be used with each treatment device 10. As seen in FIG. 3, sets of holes or openings 54 and 56 are provided to support and align the outer tubes 20 and bulbs 14, respectively.

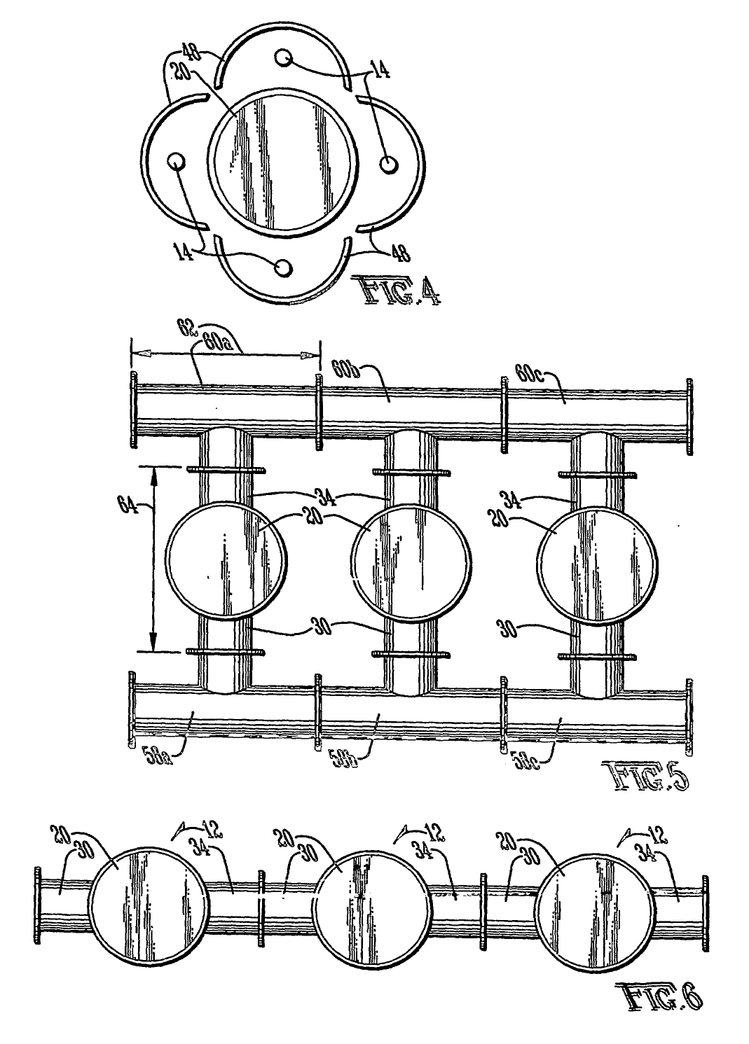

Referring to FIG. 5, input and output manifolds 58 and 60 are provided and are disposed to allow for parallel flow of a liquid through a plurality of adjacent treatment chambers 12. The manifolds are provided in a modular arrangement with a first set of associated input and output manifold segments 58a and 60a, a second set of associated input and output manifold segments 58b and 60b, and so on for the desired number of treatment chambers 12 to be used. The length 62 of the each input and output manifold 58, 60 segment is equal to the distance 64 between the input opening 32 of the input pipe 30 and the output opening 36 of the output pipe 34. This allows each treatment chamber 12 to be quickly and easily adjusted to provide for either parallel flow as seen in FIG. 5 or to provide for series flow as seen in FIG. 6.

FIG. 6 shows a plurality of treatment chambers 12 arranged to provide for series flow through a plurality of treatment chambers 12. In this arrangement, the output opening 36 of an output pipe 34 of a first treatment chamber 12 is aligned with an input opening 32 of an input pipe 30 of a second treatment chamber 12, and so on for the desired number of treatment chambers 12.

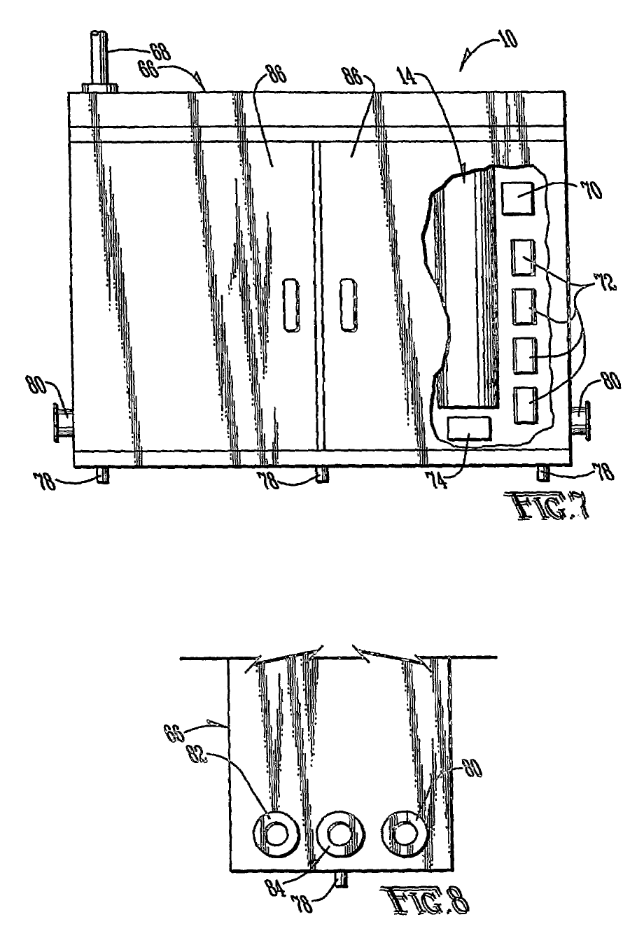

As shown in FIG. 7, a radiation treatment device 10 of the present invention may also include a cabinet 66 and related components. One or more treatment chambers 12 and sets of associated bulbs 14, reflectors 48, and input and output manifolds 58, 60 are housed within the cabinet 66. The cabinet 66 is preferably made primarily of stainless steel. Other components may be disposed within or positioned near the cabinet 66. For example, a power line 68 may supply power to controls 70 and to ballast 72 associated with each bulb 14, which may be housed in the cabinet 66 or separately above the cabinet 66. A fan 74 may be provided for cooling the ballast 72 and controls 70, and drain pipes 78 may be provided in the cabinet 66 floor. In the preferred embodiment, a separate fan 74 will be associated each modular illumination unit 46, with the fan 74 disposed to provide a positive pressure cabinet. It is of course understood that any number of different fan 74 arrangements may be used and that one or more fans may be disposed to provide either a positive pressure cabinet or a negative pressure cabinet. One or more input or output pipes 80, 82, and 84 may be provided, disposed in lower side walls of the cabinet 66. As best seen in FIG. 8, outer pipes 80 and 82 are disposed to align with input and output manifolds 58 and 60, respectively, to provide a path for parallel flow of liquid through the treatment chambers 12 such as when the treatment chambers 12 are aligned as depicted in FIG. 5 . The centrally located pipes 84 are disposed to align with input and output pipes 30 and 34 of the treatment chambers 12 when the treatment chambers 12 are aligned for series flow, such as seen in FIG. 6.

Referring to FIGS. 5 and 6, in operation, a plurality of treatment chambers 12 are aligned as desired to provide for parallel or series flow through the desired number of treatment chambers 12. It is of course understood that a single treatment chamber 12 may also be used if desired. Once the treatment chambers 12 are aligned as desired and the cabinet doors 86 closed for added protection against exposure to UV radiation, the bulbs 14 are activated to provide UV radiation. The liquid to be treated is then provided to the device 10 at the desired pressure and flow rate. It is understood that the device 10 may be used in connection with most any liquid, including but not limited to clear or opaque liquids such as water, including wastewater, juices, brines, marinades, beverages, and the like.

In parallel flow (FIG. 5) the liquid will pass through and fill the desired number of input manifold segments 58a, 58b, 58c and will pass from each input manifold 58 segment into an associated treatment chamber 12. As best seen in FIG. 1, the liquid passes through the input pipe 30, through the housing 26, and into the annulus 44 between the inner tube 18 and outer tube 20. The static mixer 22 routes the liquid in a tight spiral pattern along a helical path upward through the annulus 44 to an upper portion of the treatment chamber 12. As the liquid passes through the narrow annulus 44 in close proximity to the bulbs 14, UV radiation from the bulbs 14 provides the desired degree of disinfection. The use of the auger style static mixer 22 provides for significant mixing and churning of the liquid as it passes upward through the annulus 44 so that different portions of the liquid are constantly being moved closer to and further from the bulbs 14. This ensures thorough and even radiation exposure throughout the liquid and greatly reduces the chances of leaving isolated portions relatively untreated or significantly over-treated. The end cap 24 arrests upward flow of the liquid and redirects the liquid to flow downward through the inner tube 18. The liquid then passes through the inner tube 18, through the inner header tube 28, and through the output pipe 34. If the treatment chamber 12 is aligned to provide for parallel flow (FIG. 5), the liquid passes from the output pipe 34 to and through the associated output manifold 60 segment for further use or treatment. If the treatment chamber 12 is aligned to provide for series flow (FIG. 6), the liquid passes from the output pipe 34 of one treatment chamber 12 to the input pipe 30 of another treatment chamber 12 to repeat the process described above.

The rugged device 10 of the present invention may be operated under wide ranges or pressures and flow rates without fear of damaging the device 10. For example, the device 10 of the present invention may be safely operated at a working pressure reaching or exceeding a pressure that is preferably substantially within a range of from approximately 30 psig to approximately 60 psig and that is more preferably approximately 57 psig. The device 10 may withstand burst pressures reaching or exceeding a pressure that is preferably substantially within a range of from approximately 100 psig to approximately 300 psig and that is more preferably approximately 286 psig. Desired flow rates for many applications will typically be within a range of from approximately 1 gallon per minute to approximately 20 gallons per minute. Similarly, desired flow rates for typical clean in place cleaning will typically be less than or equal to approximately 25 gallons per minute. Still, much higher flow rates may be desirable for some applications, such as for the batch processing of juice. In the batch processing of juice, it is sometimes desirable to process flow rates reaching or exceeding approximately 70 gallons per minute. Because of limitations imposed by the relatively fragile nature of prior radiation treatment devices, it is not believed that UV radiation treatment has been used in applications calling for such high flow rates. In contrast, the rigid construction of the present invention will preferably allow the present invention to safely process flows rates of up to approximately 30 gallons per minute, will more preferably allow the present invention to safely process flows rates of up to approximately 55 gallons per minute, and will most preferably allow the present invention to safely process flows rates of up to approximately 80 gallons per minute. A treatment chamber 12 typically processes approximately 10 to 12 gallons per minute. Parallel flow is typically used for higher rates.

Other modifications, changes and substitutions are intended in the foregoing, and in some instances, some features of the invention will be employed without a corresponding use of other features. For example, any number of treatment chambers 12 may be used, from one to several. Similarly, although it is preferred to use a configuration of eight bulbs 14 per treatment chamber 12, any number of bulbs 14 may be used in connection with a treatment chamber 12, from one to several. Also, any number of different types of mixers 22 may be used in the annulus 44, or a mixer 22 may be omitted. Further, any number of different flow paths may be used, including but not limited to a flow path that is roughly the reverse of that described in the preferred embodiment. Similarly, strictly series flow may be used, strictly parallel flow may be used, or any number of combinations of series and parallel flows may be used. Also, the header 16 may be disposed in different locations, such as at the top of the treatment chamber 12. Similarly, any number of different methods may be used to route the fluid to or from the annulus 44 area and to or from the inner tube 18. Although bulbs 14 providing UV radiation are preferred, any number of different types of radiation and types of radiation sources 14 may be used depending upon the desired application. Further, the reflectors 48 may take any number of shapes, sizes or configurations or may be omitted. Further still, any number of different structures and arrangements may be used for supporting and aligning the various components of the device. Similarly, any number of different structures and arrangements may be provided for shielding users and surrounding environments from radiation exposure. Although the preferred embodiment is particularly useful for treating liquids, it is of course understood that the invention may be used in connection with treating any number of different forms of matter. For example, a device of the present invention may also be used to treat a gas or to treat fluid matter, including but not limited to solid particulate matter. It is of course understood that all quantitative information is given by way of example only and is not intended to limit the scope of the present invention.

What is claimed is:

1. An apparatus, comprising:
an outer tube having an inner surface;
an inner tube having an inner surface and an outer surface, said inner tube being disposed within said outer tube so that said inner surface of said outer tube and said outer surface of said inner tube define an outer flow path and so that said inner surface of said inner tube defines an inner flow path, said inner tube having an opening, said opening placing said outer flow path in fluid flow communication with said inner flow path; and
a radiation source disposed adjacent to said outer tube.

2. The apparatus of claim 1, further comprising:
a helical member disposed within said outer flow path.

3. The apparatus of claim 1, further comprising:
a header, said header being affixed to a proximal end portion of said outer tube and to a proximal end portion of said inner tube, said header being in fluid flow communication with said outer flow path and said inner flow path.

4. The apparatus of claim 3, wherein said header defines an input flow path and a separate output flow path, said input flow path being in fluid flow communication with said outer flow path or said inner flow path and said output flow path being in fluid flow communication with the other of said outer flow path or said inner flow path.

5. The apparatus of claim 3, wherein said header defines an input flow path and a separate output flow path, said input flow path being in fluid flow communication with said outer flow path and said output flow path being in fluid flow communication with said inner flow path.

6. The apparatus of claim 1, further comprising:
an end cap affixed to a distal end portion of said outer tube.

7. The apparatus of claim 6, wherein said opening of said inner tube is disposed at a distal end portion of said inner tube.

8. The apparatus of claim 1, wherein said inner tube is not transparent with respect to radiation from said radiation source.

9. The apparatus of claim 1, wherein said inner tube is comprised of stainless steel.

10 An apparatus, comprising:
a treatment chamber; and
a radiation source disposed in close proximity to said treatment chamber, said radiation source comprising:
a first bulb disposed near said treatment chamber;
a second bulb disposed near said treatment chamber;
first reflector segment disposed near said first bulb, said first reflector segment having a first cross section; and
a second reflector segment adjacent said first reflector segment and being disposed near said second bulb, said second reflector segment having a second cross section in a common plane with said first cross section, said first cross section and said second cross section not forming portions of a common circle or semi-circle.

11. The apparatus of claim 10, wherein said first cross section is semi-circular and said second cross section is semi-circular.

12. The apparatus of claim 10, wherein said first cross section has a first arc length that is greater than approximately 45°.

13. The apparatus of claim 12, further comprising:
a first bracket disposed near a first portion of said treatment chamber; and
a second bracket disposed near a second portion of said treatment chamber, said first, second, and third reflector segments being affixed to said first bracket.

14. The apparatus of claim 13, further comprising:
a fourth bulb disposed near said treatment chamber;
a fifth bulb disposed near said treatment chamber;
a fourth reflector segment disposed near said fourth bulb, said fourth reflector segment having a fourth cross section; and
a fifth reflector segment adjacent said fourth reflector segment and being disposed near said fifth bulb, said fifth reflector segment having a fifth cross section in a common plane with said fourth cross section, said fourth cross section and said fifth cross section not forming portions of a common circle or semi-circle.

15. A method, comprising:
(1) providing a first tube and a second tube, said second tube being disposed at least partially within said first tube;
(2) passing a liquid between an inner wall of said first tube and an outer wall of said second tube;
(3) irradiating said liquid as said liquid passes between said inner wall of said first tube and said outer wall of said second tube; and
(4) before or after step (2), passing said liquid through an interior of said second tube.

16. The method of claim 15, wherein step (4) comprises: after step (2), passing said liquid through said interior of said second tube.

17. The method of claim 15, wherein step (2) comprises: passing said liquid along a helical path between said inner wall of said first tube and said outer wall of said second tube.

18. The method of claim 17, wherein (3) comprises: irradiating said liquid with a UV bulb as said liquid passes between said inner wall of said first tube and said outer wall of said second tube.

19. The method of claim 15, wherein step (2) comprises: passing said liquid between said inner wall of said first tube and said outer wall of said second tube at a pressure that is greater than or equal to approximately 30 psig.

20. The method of claim 15, wherein step (2) comprises: passing said liquid between said inner wall of said first tube and said outer wall of said second tube at a flow rate that is greater than or equal to approximately 10 gallons per minute.
